(19) United States
(12) Patent Application Publication
(10) Pub. No.: US 2022/0096328 A1
(43) Pub. Date: Mar. 31, 2022

(54) Apparatus and method for buffering solutions

(71) Applicant: Scott Papineau, Fishers, IN (US)

(72) Inventor: Scott Papineau, Fishers, IN (US)

(21) Appl. No.: 17/033,703

(22) Filed: Sep. 26, 2020

(51) Int. Cl. A61J 1/20; A61J 1/14

(52) U.S. Cl. CPC A61J 1/2096; A61M 2202/048; A61J 1/1406

(57) ABSTRACT

The present invention is directed to a method an apparatus for buffering an anesthetic solution prior to an injection. The inventive apparatus employs a modified carpule which hold two solutions in fluid isolation. The buffering solution is separated from the anesthetic solution within the same carpule by way of a fluid impervious thin film. The thin film has a weakened central area which can be easily pierced by a harpoon affixed to one side of a plunger. As the plunger is advanced forward, the harpoon breaks through the thin film and causes mixing of the two solutions.

### BACKGROUND OF THE INVENTION

### 1. Field of the Invention

The present invention relates generally to methods and apparatus for buffering anesthetics.

More particularly, the present invention relates to methods for preparing and storing sodium bicarbonate buffering solutions and combining such solutions with anesthetics stored in small cartridges.

Aqueous solutions containing bicarbonate ions are used in various medical applications. The present application concerns bicarbonates solutions that are used to buffer dental and other anesthetics to control pH.

### 2. Description of the Prior Art

It is known that the storage abilities of a number of pharmaceutical compositions are directly linked to their basic pH, and that a buffering process has to be implemented extemporaneously shortly prior to injection. Particularly, the issue of buffering a composition to be injected, using a standard apparatus for injecting has been addressed by many prior art apparatuses. This is a constant issue in the field of dental anesthetics.

US patent application 2009/221,984 describes a method and apparatus for buffering lidocaine (with and without epinephrine) while improving shelf life without the need to refrigerate. Neutralizing the pH of dental anesthetics prior to injection may also improve efficacy of these anesthetics, while lowering the pain of the patient during injection and following minutes.

United States Patent Application 20090292271 discloses a device for delivering buffer solution into a buffer cartridge comprises a needle assembly and a pusher. The septa of a buffer cartridge and an anesthetic cartridge are advanced onto a transfer needle, and the pusher advances a plunger into the buffer cartridge to deliver a buffer solution through the transfer needle into the anesthetic cartridge. A separate exhaust needle allows excess anesthetic from the anesthetic cartridge to be exhausted. A compression member is usually provided to maintain a force against the plunger on the buffer cartridge to pressurize and stabilize the contents during sterilization and/or storage. This device suffers from the drawback in that it requires a substantial modification of the syringe/plunger assembly, resulting in a new device which requires a learning curve just to utilize.

While these and other prior art methods are generally effective for their intended purposes, they suffer from the drawback that they require substantial modifications of the existing syringe/plunger systems to be effective.

The present invention is directed to a method an apparatus for buffering an anesthetic solution prior to an injection. The inventive apparatus employs a modified carpule which holds two solutions in fluid isolation. The buffering solution is separated from the anesthetic solution within the same carpule by way of a fluid impervious thin film. The thin film has a weakened central area which can be easily pierced by a harpoon affixed to one side of a plunger. As the plunger is advanced forward, the modified harpoon breaks through the thin film and causes mixing of the two solutions.

### SUMMARY OF THE INVENTION

It is a major object of the invention to provide a method and apparatus for buffering solutions.

It is another object of the invention to provide a method and apparatus for buffering anesthetics immediately prior to use.

It is another object of the invention to provide a method and apparatus for buffering anesthetics which uses a modified carpule.

It is another object of the invention to provide a method and apparatus for buffering anesthetics which employs a modified plunger.

### BRIEF DESCRIPTION OF THE DRAWINGS

FIG. 1 shows a side sectional view of the inventive apparatus.

FIG. 2 shows side and plan views, partly in section, detailing the modifications of the present invention.

FIG. 3 shows a side view of a syringe with the inventive apparatus positioned therein.

### DETAILED DESCRIPTION OF THE PREFERRED EMBODIMENTS

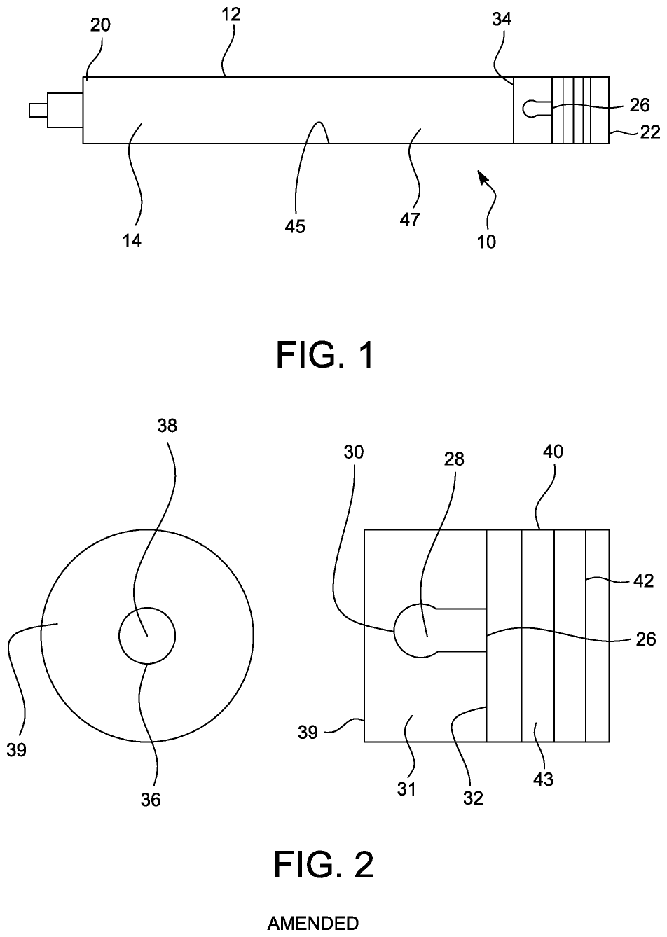

Referring now to the drawings, FIG. 1 illustrates the inventive apparatus, generally indicated by the numeral 10. The apparatus 10 has the ability to mix, just prior to use, an anesthetic and a buffering solution. The apparatus 10 is essentially a carpule mixing assembly, the carpule and internal plunger of the present invention being modified to perform the mixing of the two solutions with a single depression of the syringe plunger. The term “carpule” refers to a container, such as a vial, cartridge, or the like, generally made of glass and adapted to house a dose of a medical fluid. The carpule can be inserted into a syringe 11 for dispensing (e.g., injecting) into a patient. Prior art carpules typically include a puncturable cap on one end and a sliding plug on the other end. The cap can be punctured by the tip of a needle assembly of a carpule syringe to allow the fluid housed within the carpule to be dispensed. The plug is advanced towards the cap end of the carpule via a syringe plunger.

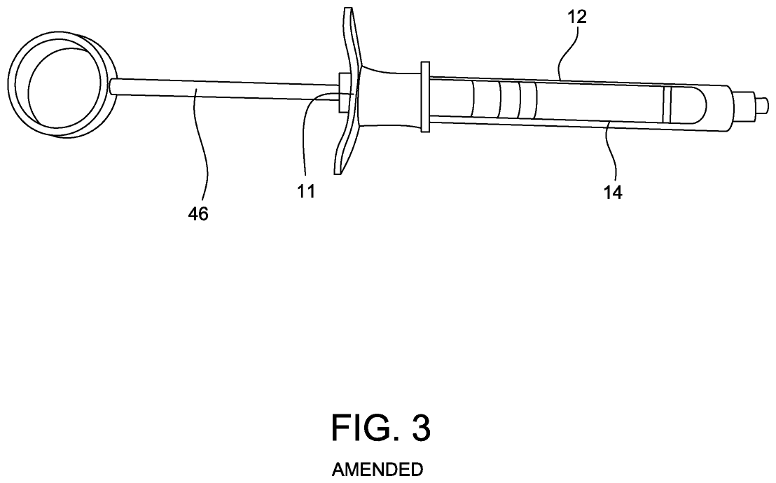

FIG. 1 illustrates the modified carpule of the apparatus 10. As shown the carpule 12 is configured in a cylindrical shape with hollow interior 14 and open ends 20, 22. Of course, the carpule 12 can have any desired shape. The carpule 12 is designed for positioning within a standard syringe 11 as shown in FIG. 3. The carpule 12 contains two medications, the first may be a buffering solution contained in a first fluidly isolated compartment, and the second may be an anesthetic contained within a second fluid filled compartment which needs to be buffered just prior to use. While the invention is described as a method of mixing anesthesia with a buffer solution, within the scope of the invention, any fluids that need to be mixed upon injection may be contained within the carpule 12.

The first and second fluid filled compartments are positioned within the hollow interior 14 of the carpule 12, and are dispensed in mixed relation as a result of the movement of an internal sealing plug or plunger 26 from first end 20 to second end 22. The carpule 12 can be constructed from any desired material, such as (but not limited to) glass, polymeric material, ceramic material, metal (e.g., stainless steel), or combinations thereof, but the material is at least partially transparent to allow the user to monitor mixing of the first and second solutions.

With reference now to FIGS. 1-3, it can be seen that the plunger 26 has a projection 28 extending therefrom, the projection 28 having a pointed tip 30. The projection 28 extends into a cylindrical fluid filled space or first compartment 31 defined by the sealing face 32 of the modified plunger 26 of the invention on one end, and a circular fluid barrier 34 formed at the other end. The fluid barrier 34 is positioned in slidable sealing relation within the carpule 12 to create the first and second compartments. Fluid barrier 34 may be formed of a rigid material such as hard plastic, the barrier including a central aperture 36 covered by an easily pierced thin film 38. The thin film 38 may be made of plastic, metal foil, or equivalent material as would be apparent to one of skill in the art.

The modified plunger 26 includes a main body 40 which is inserted into the carpule 12 in sealing relation therewith. The plunger 26 has a plug portion 42 which is axially slidable movement within the main body 40 in response axial force exerted thereon by plunger handle 46. The circular outer diameter 43 of the plug portion 42 is sized for sliding and sealing engagement with the inner wall 45 of the first compartment 31. Axial movement of the plug portion 42 relative to fluid barrier 34 forces projection 28 into the thin film 38 resulting in the rupturing thereof and the mixing of the solution contained therein with the solution contained within the second compartment 47, thereby effecting the objective of the invention. Axial movement of the plug portion 42 is effected by axial movement of the syringe plunger 46.

In use, with the modified plunger 26 of the invention inserted within a carpule 12, the carpule 12 positioned within the syringe 46, the e.g., dentist simply depresses the plunger handle 46 of the syringe, effecting axial movement of the plug portion 42 and urging projection 28 into thin film 38 to effect the buffering of the anesthetic contained therein by mixing the solutions of the first and second compartments.

It is to be understood that the present invention is not limited to the sole embodiment described above, but encompasses any and all embodiments within the scope of the following claims:

I claim:

1. A modified carpule apparatus for a syringe used to mix two solutions comprising;
a generally cylindrical main body having an axially slidable plug contained therein, said main body having first and second fluid filled hollow portions separated by a seal with a piercable center;
a projection extending from said plug, wherein axial movement of said plug can cause piercing of said center releasing fluid from said first hollow portion into said second hollow portion.

2. The apparatus of claim 1 wherein said seal has a rigid outer portion.

3. The apparatus of claim 2 wherein said outer portion is formed of a plastic material.

4. The apparatus of claim 1 wherein said first and second hollow portions have inner walls and said plug has an outer diameter in sealing relation with said inner walls.

5. The apparatus of claim 1 wherein said piercing of said seal causes fluid to flow from said first fluid filled hollow portion to said second fluid filled hollow portion.

6. The apparatus of claim 5 wherein said first hollow portion contains a first medication and said second hollow portion contains a second medication.

7. The apparatus of claim 6 wherein said first medication is a buffering solution and said second medication is an anesthetic.
